(19) United States
(12) Patent Application Publication
(10) Pub. No.: US 2003/0018340 A1
(43) Pub. Date: Jan. 23, 2003

(54) Method and apparatus for installing cannula

(76) Inventor: Thomas P. Branch, Atlanta, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/186,340

(22) Filed: Jun. 28, 2002

Related U.S. Application Data
(60) Provisional application No. 60/301,982, filed on Jun. 29, 2001.

(51) Int. Cl. A61B 17/00

(52) U.S. Cl. 606/96; 606/190

(57) ABSTRACT

A system is disclosed for stabilizing a cannula or tube which provides access into the body during a laparoscopic or an arthroscopic procedure. One or more fastening tubes or cannula are attached adjacent to a central tube or working cannula. Elongate fastening members are passed through the fastening cannula and are attached to bone which stabilizes the working cannula. This stabilization allows the surgeon to operate through the working cannula without concern that the cannula apparatus will be displaced. Furthermore, one of the wires can be detached and the cannula system rotated about the remaining secured fastener with the first fastener being re-attached at a new location. This technique allows the surgeon to place implants at uniform distances from one another without the cannula apparatus losing contact with the bone.

### CROSS-REFERENCE TO RELATED APPLICATION

This application claims the full benefit and priority of pending prior application No. 60/301,982, filed Jun. 29, 2001, entitled METHOD AND APPARATUS FOR INSTALLING CANNULA, and incorporates said application by reference.

### FIELD OF THE INVENTION

The present invention relates generally to medical devices, and more particularly relates to the use of medical devices and/or implant procedures which include the use of cannulae.

### BACKGROUND OF THE INVENTION

Laparoscopy and Arthroscopy are well known techniques used by surgeons for diagnosing and repairing damaged or diseased areas within the body such as the abdomen, knee, wrist, hip, etc. These minimally invasive techniques facilitate treatment without requiring large incisions. For either procedure, a series of relatively small holes or ports are created in the skin. A cannula or tube is inserted into these incisions to provide access to the treatment area by the surgeon. A fiber optic video camera or endoscope is inserted into one of the cannulae for viewing the treatment area inside the body. Simultaneously, the surgeon can insert specially designed tools through auxiliary cannulae to probe or repair the damaged or diseased area while viewing through the endoscope. This technique's use of small incisions reduces the chance of complications and the recovery time for the patient. While the surgeon is viewing or repairing an area, the cannula can become displaced due to inadvertent contact with a surgical tool or the endoscope. As a result, the surgeon must interrupt the procedure to replace the cannula or someone must hold the cannula in place during the procedure. FIGS. A-C located in the Appendix are photos depicting a prior art “Bionxx” arrow system which is used to implant a medical device into a torn meniscus. The system comprises a main cannula with a pointed portion or tooth on the distal end and a pointed side wire passing through a smaller cannula attached to the side of the main cannula. The pointed portion or tooth of the main cannula and the pointed wire engage soft tissue to stabilize the cannulae. However, the side wire is provided with a small handle on its end that does not allow the wire to be drilled into the bone. Also, the pointed tooth of the main cannula cannot be driven into bone for firm fixation without damaging the soft tissue by a knife action. Furthermore, with only one fixation cannula in addition to the working cannula there is limited opportunity for obtaining optimum fixation when the working cannula is placed in its optimum position. In other words, the anatomy of the human bone has ridges and shapes that may not allow firm fixation with the wires at 1 o'clock and 7 o'clock but will allow firm fixation with the wires in other positions around the working cannula.

Thus, there is a need during laparoscopic or arthroscopic surgery to anchor a working cannula in place in its optimum position into bone. This would allow the surgeon to let go of the cannula and not be concerned that it would be displaced.

### SUMMARY OF THE INVENTION

The present invention seeks to provide a cannula anchoring system that can stabilize a cannula during a laparoscopic or an arthroscopic procedure. The present invention accomplishes this objective by providing a cannula apparatus with one or more elongate cavities through which elongate fastening members may pass. These fasteners engage bone and thereby stabilize the cannula apparatus for use by the surgeon.

Generally described, the present invention provides a cannula apparatus for removeably attaching a working cannula to bone. The cannula apparatus comprises a working cannula which provides access to a treatment area through an elongated cannula passageway; a cavity defining portion which defines one or more elongated cavities adjacent to the working cannula passageway extending to a position adjacent to the bone when the working cannula is positioned at the treatment area, and an elongate fastening member passing through the cavity. The fastening members are adapted to be removeably attached to the bone. The fastening member may comprise a wire with a proximal end and distal end, wherein the distal end includes a self-tapping configuration or a diamond shaped tip.

In a preferred embodiment of the invention, the cavity defining portion comprises one or more fastening cannula adjacent to the working cannula. The fastening cannula may be smaller than the working cannula. In a further embodiment, the working cannula has an inner surface and an outer surface and the cavity defining portion forms one or more elongated cavities between these inner and outer surfaces. Preferably, two or more cavities are defined for receiving fastening members capable of engaging bone.

According to another of its aspects, the present invention provides a method for using a cannula apparatus including a plurality of fasteners in conjunction with a bone, comprising attaching a first fastener to the bone; attaching a second fastener to the bone; detaching the first fastener; and rotating the cannula apparatus about the second fastener. In one implementation, the first fastener may be re-attached to the bone at a different location.

### BRIEF DESCRIPTION OF THE DRAWINGS

FIG. 1 is a pictorial view of cannula apparatus embodying the present invention positioned adjacent to a shoulder joint.

FIG. 2 is a pictorial view of a cannula apparatus embodying the present invention. An installation tool is shown in phantom therewith.

FIG. 3 is a side plan view of one of the wire members shown in FIG. 2.

FIG. 4 is a pictorial view of an alternative embodiment of the present invention.

FIG. 5 is a pictorial view of a third embodiment of the present invention.

FIG. 6 is a partially exploded pictorial view of a fourth embodiment of the present invention.

FIG. 7 is a cross-sectional view taken along line 2-2 of FIG. 6.

### DETAILED DESCRIPTION

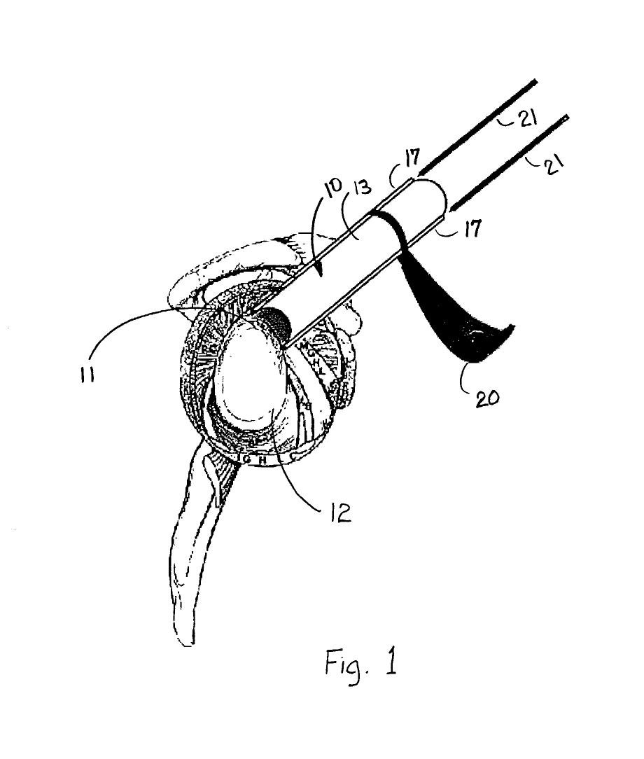

Referring now to the drawing, in which like numerals refer to like parts throughout the several views, FIG. 1 shows a cannula apparatus 10 embodying the present invention positioned next to a shoulder joint. The cannula apparatus is inserted though an incision in the skin and provides access to the treatment area 11. The cannula apparatus is shown adjacent to a bone 12 which in FIG. 1 is the anterior glenoid of the shoulder. The cannula apparatus is shown with a handle 20 strapped around its outer surface. The handle 20 is an installation tool used to position the cannula in the incision prior to attaching it to the bone 12.

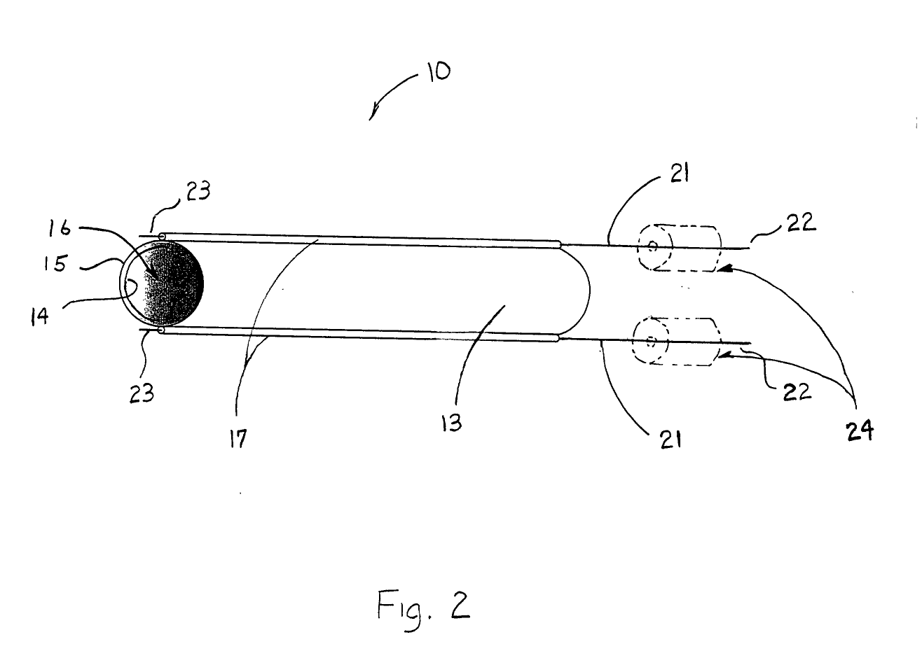

Referring to FIGS. 1 and 2, the cannula apparatus 10 includes a working cannula 13 which has a tubular structure defined by an inner surface 14 and an outer surface 15. The inner surface further defines a central passageway 16 through which the surgeon accesses the treatment area 11. The working cannula in the preferred embodiment is approximately 150 mm long and the passageway 16 has a circular opening of approximately 7 mm in cross-section. Other cross-sectional shapes and sizes may be used. The cannula apparatus 10 may be constructed of metal or plastic, and may be opaque or transparent.

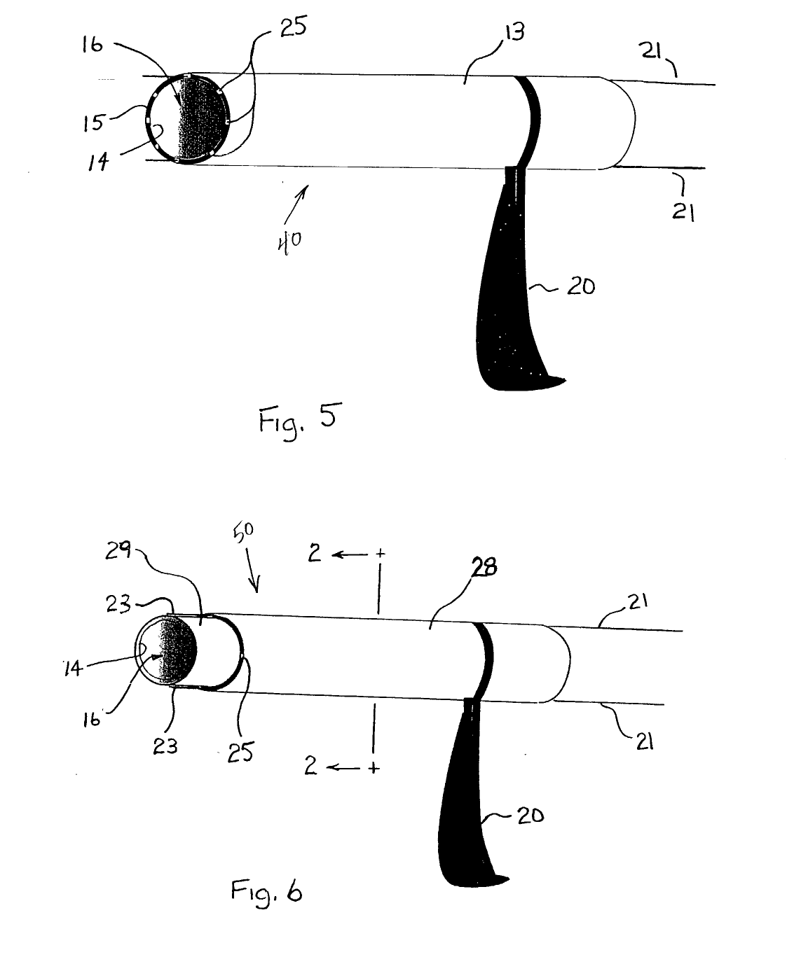

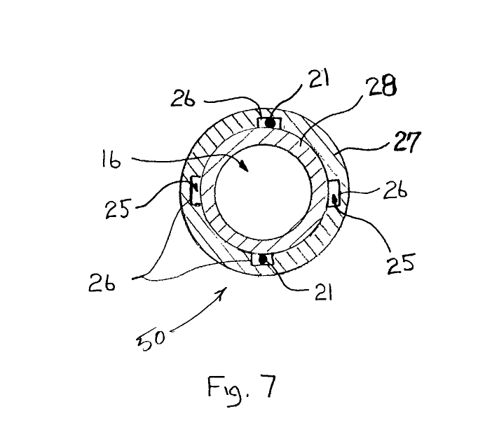

The cannula apparatus 10 also includes one or more side cannulae or fastening cannulae 17 attached to the outer surface 15 of the working cannula 13. In the preferred embodiment, the fastening cannulae define an elongate cavity and are generally round in transverse cross-section with an inner diameter of approximately 0.045 mm. It is also envisioned that numerous fastening cannulae could be attached to a working cannula of a cannula apparatus 30 as shown in FIG. 4. While the cross-sections of the fastening cannulae are generally round, this need not be the case as is shown in FIG. 5. In this embodiment of a cannula apparatus 40, the elongated cavities or slots 25 are formed in the walls of the working cannula. Slots can also be formed using a two piece construction as shown in the cannula apparatus 50 of FIGS. 6 and 7. This two piece construction provides channels 26 formed in the inner surface of the lumen of an outer sleeve 27. An inner sleeve 28 is inserted into the outer sleeve thereby enclosing the channels 26, forming slots 25. It should be understood that while the embodiments shown in FIGS. 5, 6, and 7 only show four slots to simplify the description of the invention, the invention includes providing one, two, or many slots adjacent to the cannula passageway 16.

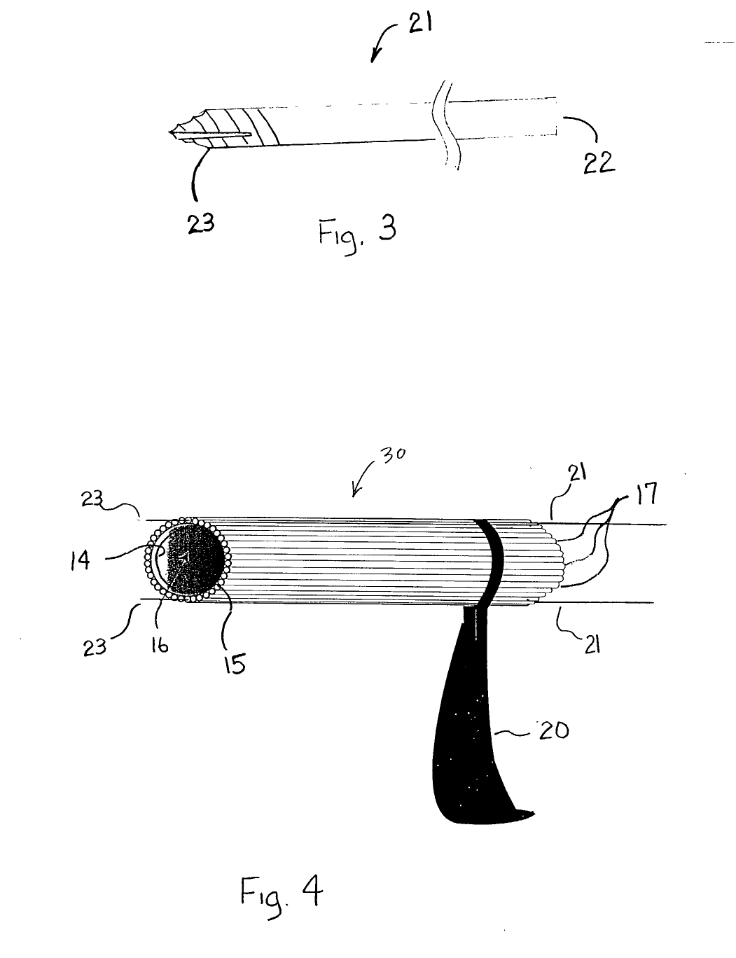

In all the embodiments discussed above, the fastening cannulae or slots are sized to allow the insertion of a wire member 21 into one or more of the cavities. Referring to FIG. 3, the wire members 21 have a proximal end 22 and a distal end 23. The distal ends are formed with a self-tapping configuration or a conventional plain diamond shaped tip. In operation, the distal end of the wire members are threaded through the fastening cannulae 17 or slots 25 and tapped into the bone. The tapping action of the wire is accomplished by simply twisting the wire member by hand or by using a conventional wire gripping device 24 to gain additional torque. A motor-driven rotatory device such as a drill could be used. It is also envisioned that the wire members could be bowed away from the working cannula 13 to provide unobstructed access to the cannula passageway 16 while still allowing the rotation of the wire member. The wire members in the preferred embodiment may be “k-wires” or other suitable elongate fastening devices which allow for engagement of bone. The k-wires may be tipped with threaded self-tapping ends as described above or with conventional diamond shaped tips that may be drilled into bone where they are held by friction. Both distal end configurations described lock the wire into bone.

Operation of the cannula apparatus 10 is described as follows, with particular reference to FIGS. 1 and 2. A surgeon makes a small incision in the skin using a scalpel or trocar (not shown) and the cannula apparatus is inserted into the incision. The distal ends 23 of the wire members 21 are then threaded through the fastening cannulae 17 or slots 25 until they contact the bone 12. A clockwise torque is applied to each wire member along with a slight axial force to urge the self tapping or diamond shaped distal end of the wire members to engage the bone. Once the wire members are engaged into the bone, the cannula apparatus is stabilized and the surgeon is free to access the treatment area through the cannula passageway 16 without the concern of displacing the cannula apparatus. The surgeon will typically insert a fiber optic video camera (not shown) through the cannula passageway to diagnose the treatment area 11 and may use specially designed tools (not shown) inserted though the cannula passageway to repair the damaged or diseased area.

The embodiments which employ a plurality of cannulae or slots, as shown in FIGS. 4 and 5 for example, provide the surgeon with the option of multiple attachment locations without having to move the cannula apparatus. The surgeon has the flexibility to choose which cannulae to place fasteners in and how many attachment points are necessary for a particular procedure.

The embodiment shown in FIGS. 1 and 2 can also be used to apply implants at uniform distances from each other. In this technique, the cannula apparatus 10 is inserted into an incision and two wire members 21 are attached to the bone 12 as described above. After an implant is applied through the cannula passageway 16, one of the wire members is removed from the bone by twisting the wire counter-clockwise. The cannula apparatus is then rotated about the single secured wire until the desired location is reached. The non-secured wire member is then reattached at this new location. This technique allows the surgeon to “walk” the cannula apparatus about the treatment area and maintain a uniform distance between implants without losing contact with the bone.

A further method of use for the present invention is to manipulate soft tissue within the body. In this method, the distal ends 23 of the wire members 21 are extended though the fastening cannula 17 or slots 25 beyond the end of the working cannula 13 and are used to pierce soft tissue. The cannula apparatus 10 is then moved until the tissue is in the desired location and the wire member is retracted from the soft tissue. Similarly, soft tissue can be manipulated with one of the wire members secured to bone. The non-secured wire members pierce the soft tissue and then the assembly is rotated about the secured wire member until soft tissue is in the desired location. At that point, the non-secured wire members are retracted from the soft tissue. The ends of the wire can drill into soft tissue without any damaging knife action. Prior systems such as shown in the Appendix are not capable of similar manipulation of tissue.

Thus, it will be seen that the present cannula apparatus and methods are superior to prior art systems and methods in that various embodiments of the present apparatus can be locked into bone by drilling, does not tend to slice tissue by a knife action during installation, can provide a plurality of fastening wires associated with a main cannula as well as a selection of positions in which to use the fastening wires, can provide fastening wire passageways built into the main cannula wall if desired, and have the other advantages described above.

While this invention has been described in detail with particular reference to preferred embodiments thereof, it will be understood that variations and modifications can be effected within the spirit and scope of the invention as described hereinbefore and as defined in the appended claims.

[image]

[image]

[image]

[image]

That which is claimed:

1. A cannula apparatus for removeably attaching a working cannula to bone, said cannula apparatus comprising:
a working cannula providing access to a treatment area through an elongated cannula passageway;
a cavity defining portion defining one or more elongated cavities adjacent to said working cannula passageway extending to a position adjacent to said bone when said working cannula is positioned at said treatment area; and
an elongate fastening member passing through said cavity and adapted to be removeably attached to said bone.

2. A cannula apparatus as described in claim 1, wherein said cavity defining portion comprises a fastening cannula attached adjacent to said working cannula.

3. A cannula apparatus as described in claim 2, wherein a plurality of spaced apart fastening cannula are attached to said working cannula.

4. A cannula apparatus as described in claim 1, wherein said working cannula has an inner surface and an outer surface, said cavity defining portion forms said elongated cavity between said inner and outer surfaces of said working cannula.

5. A cannula apparatus as described in claim 4, wherein a plurality of spaced apart cavities are formed between said inner and outer surfaces.

6. A cannula apparatus as described in claim 1, wherein the cannula apparatus is formed from plastic.

7. A cannula apparatus as described in claim 1, wherein the cannula apparatus is formed from a metal.

8. A cannula apparatus as described in claim 1, wherein said elongate fastening member comprises a wire with a proximal end and distal end, said distal end including a self tapping configuration.

9. A cannula apparatus removeably attaching a working cannula to bone, said cannula apparatus comprising:
a working cannula providing access to treatment area, having an inner and an outer surface;
one or more fastening cannulae adjacent to said working cannula; and
one or more wire members adapted to be received within said fastening cannula, said wire having a proximal end and distal end, said distal end including a self tapping configuration.

10. A cannula apparatus as described in claim 9, wherein said fastening cannula is smaller in diameter than the working cannula

11. A cannula apparatus as described in claim 9, wherein a pair of fastening cannulae are located adjacent to said working cannula and are spaced apart.

12. A cannula apparatus as described in claim 9, wherein said cannula apparatus is formed from plastic.

13. A cannula apparatus as described in claim 9, wherein the cannula apparatus is formed from a metal.

14. Method for using a cannula apparatus including a plurality of fasteners in conjunction with a bone, comprising:
attaching a first fastener to said bone;
attaching a second fastener to said bone;
detaching said first fastener; and
rotating said cannula apparatus about said second fastener.

15. Method as described in claim 14, further comprising:
re-attaching said first fastener to said bone at a different location.
